(19) United States
(12) Patent Application Publication
(10) Pub. No.: US 2014/0335173 A1
(43) Pub. Date: Nov. 13, 2014

(54) RELEASE CAPSULES, MANUFACTURE AND USES THEREOF

(71) Applicant: BASF SE, Ludwigshafen (DE)

(72) Inventors: Robert Wengeler, Karlsruhe (DE); Jing Dreher, Limburgerhof (DE); Andrew Patrick Full, Croton-on-Hudson, NY (US)

(21) Appl. No.: 14/364,938

(22) PCT Filed: Nov. 27, 2012
(86) PCT No.: PCT/EP2012/073687

§ 371 Date: Jun. 12, 2014

Related U.S. Application Data
(60) Provisional application No. 61/569,800, filed on Dec. 13, 2011.

(30) Foreign Application Priority Data
Dec. 13, 2011 (EP) 11193201.8

(51) Int. Cl. A61K 9/48; A61K 47/02; A61K 47/10; A61K 33/20

(52) U.S. Cl. CPC A61K 9/4816; A61K 9/4891; A61K 33/20; A61K 47/02; A61K 47/10; A61K 9/4833
USPC 424/457; 424/662; 514/772; 427/2.21

(57) ABSTRACT

The present invention relates to novel release capsules, particularly for protecting moisture-sensitive materials, particularly compositions for production of chlorine dioxide. The invention also relates to methods of manufacture of such release capsules, uses of such release capsules, and products comprising them.

The present invention is concerned with the field of release capsules, particularly for protecting moisture-sensitive materials, e.g. salts and particularly compositions for production of chlorine dioxide. The invention also relates to methods of manufacture of such release capsules, and to uses of such release capsules, particularly to products comprising them.

It is frequently desired to protect chemical agents from interaction with their environment. However, these agents eventually have to be exposed to the environment such that they can exert their intended activity, for example when environmental conditions have become favourable, or when the specified action is required.

It is thus frequently tried to separate sensitive agents from a reactive environment. This can be done for example by dosing of such agents into a reactor comprising a reaction mixture only when presence of the agent is required in the reaction mixture. Alternatively, it has been tried to include agents in a packaged form in the reaction mixture, such that no further dosage of the agent is required. Such packages could be, for example, multi-part containers comprising separate ingredients of a reaction mixture in individual compartments separated mechanically from each other. After removing of the mechanical barrier separating the compartments, the ingredients can be mixed and the agent can perform its action.

In this context, it has been tried to encapsulate agents with a shell to prevent immediate contact with a reactive environment. The capsules can be in the form of core-shell-particles with one or more shell layers, or could be capsules wherein the agent is embedded in a matrix. Also, release capsules in the form of core-shell-particles have been tried wherein the core is made of a matrix having the chemical agent embedded therein. The agent is released from capsules for example by mechanical action or mechanical disintegration of the matrix and/or shell material.

A problem is that encapsulation processes for chemical agents must not themselves expose the agent to an environment leading to deterioration of the agents. For example, temperature sensitive agents should not be exposed to high temperatures during encapsulation, and moisture sensitive agents should not be exposed to water during encapsulation.

The present invention has thus set out to provide a manufacturing process for encapsulating chemical agents, wherein the manufacturing process should allow to be performed essentially in the absence of water. The manufacturing process should also allow for complete encapsulation and should also allow achieving a long protection of encapsulated contents against water even after immersion into water. Further, the invention set out to provide corresponding release capsules, uses thereof and particularly products comprising such release capsules.

According to the invention, there is thus provided a release capsule, comprising a core having an agent to be eventually released, the agent comprising or consisting of two or more different substances, the core being surrounded by a shell of a hydrophobic material, and a metal oxide material deposited in and/or on the shell, wherein the amount of metal oxide material is chosen to delay the release of 50 wt.-% of the agent (i.e. of the total weight/total content of the two or more different substances of the agent) from the capsule upon contact of the capsule with liquid water by at least 24 hours.

It has now surprisingly been found that the combination of a hydrophobic shell material and a metal oxide material deposited in and/or on the shell allows delaying the release of 50 wt.-% of an agent from the core of a release capsule upon contact of the capsule with liquid water by at least 24 hours. Preferably, the amount of metal oxide material is chosen to (a) delay the release of 75 wt.-% of the agent (i.e. of the total weight/total content of the two or more different substances of the agent) from the capsule upon contact of the capsule with liquid water by at least 24 h and, preferably, (b) delay the release of 50 wt.-% of the agent (i.e. of the total weight/total content of the two or more different substances of the agent) from the capsule upon contact of the capsule with liquid water by at least 48 h.

The term “release” is to be construed as meaning that the agent or the substances thereof respectively leaves/leave the (inner) area surrounded by and defined by the shell of hydrophobic material and enters/enter a medium surrounding the capsule, in particular a liquid or gaseous medium surrounding the capsule, e.g. liquid water. For example, a “leaving” may occur upon influx of e.g. water into the release capsule, which may lead to a dissolution of the agent to be released and its release into the aqueous environment. Depending on the used agent and any medium surrounding the capsule, a “leaving” might also occur if the medium or any compound(s) thereof penetrates/penetrate the shell of the capsule and thus enters/enter the (inner) area of the capsule, as a consequence of which the agent or one or more substances thereof might react with the medium or any compound(s) thereof, wherein one or more (further/other) substances are generated and released from the capsule afterwards. In particular, leaving may occur in the case of a diffusion process (referring to molecular diffusion) of a surrounding liquid through the shell, which may facilitate a reaction of two different substances of the core and forming a gaseous new component, which then may leave the (inner) area surrounded by and defined by the shell.

The measurement of the release of the agent(s) contained in the capsules (as described above) or its substances, respectively, is preferably performed by using a laser technology, for example: A laser beam is send to a prism immersed in a liquid, in which the release of core agent(s) or new component(s) formed by reaction of two or more core agents (as described above) is to be studied. The substances for which the release is to be measured show a dependency of the angle of diffraction at the interface between prism and liquid as function of the concentration of the substances in the surrounding liquid. This dependency can be calibrated by adding defined amounts to the liquid and measuring the light which has passed from the laser to the prism and then back to an optical cell, i.e. by measuring the returning light. After such calibration, the prism can be immersed into the liquid to be studied, capsules are added, and the release of the core agent(s) can be studied by measuring the intensity of the returning light (as described above). It is to be noted that the capsules themselves are immersed in the liquid, but do not change the angle of diffraction at the interface. According to a preferred embodiment of the invention, the influence of any substances (in particular salt ions, e.g. Na+) (being released of the core and) remaining in the aqueous solution on the angle of diffraction is measured, while any gas formed by reaction of the core agent(s) (e.g. ClO2, CO2), if applicable, will not have an effect on the measurement. Nevertheless, since the reaction of the core agent(s) is usually known, the release can be calibrated and clearly measured. Thus, the core agent(s) are preferably selected such that the reaction of the core agent(s) (as described above) is known or, respectively, can be foreseen.

The term “a core having an agent” is to be construed as meaning that the core comprises at least one, i.e. one or more (different) agents as respectively described herein. Preferably the “core” is consisting of a first and a second agent, and optionally one or more further substances, such as binder, filler or further agents. If the core comprises more than one agent, i.e. two or more different agents as described herein, the proviso(s) regarding the delay of the release (as described herein) applies/apply to all of the core's agents. Preferably, said proviso(s) applies/apply to all substances contained in the core of the capsule, in particular if all substances contained in the core of the capsule are water soluble substances.

If the core comprises more than one agent, i.e. two or more agents, wherein each agent comprises or consists of two or more different substances, it is to be understood that the aspects described herein in view of the (at least one) agent of the core preferably apply to all agents contained in the core.

Preferably, the (at least one) agent comprises or consists of two or more different substances capable of generating a (i.e. at least one) further substance upon contact with a gaseous or liquid medium, e.g. upon contact with (liquid) water, the further substance being different from the substances of the agent contained in the core of the capsule, preferably being different from all substances contained in the core of the capsule. I.e., the two or more different substances of the agent preferably are capable of generating (by chemical reaction) said further substance upon contact with a gaseous or liquid medium or any compound(s) thereof, preferably upon contact with (liquid) water. Contact with a (gaseous or liquid) medium or any compound thereof may occur (a) after release of the agent from the capsule or (b) if the medium or any compound(s) thereof penetrates/penetrate the shell of the capsule and thus enters/enter the (inner) area of the capsule (as described above).

A release capsule according to the invention wherein the agent (preferably each agent) of the core is a chlorodioxide generating agent is particularly preferred. Preferred chlorodioxide generating agents will be described below.

Preferably, the agent contained in the core of a capsule according to the invention consists of two or more different, water soluble substances. Particularly preferred is a capsule, wherein all substances contained in the core of the capsule are water soluble substances.

Preferably, the agent contained in the core of a capsule according to the invention is a solid mixture, preferably a solid, water soluble mixture.

According to one alternative embodiment of the present invention the core of the capsule comprises, in addition to one or more agents as described above, one or more further substances. According to a preferred aspect of the present invention all substances contained in the core of the capsule are water soluble substances.

Thus, according to a particularly preferred embodiment of the present invention the capsule comprises a core having one or more agents (as described above), the agent(s) comprising or consisting of two or more different, water soluble substances capable of generating one or more further substances upon contact with a gaseous or liquid medium (as described above), the agent(s) preferably being a chlorodioxide generating agent, the further substance being different from all substances contained in the core of the capsule, wherein all substances contained in the core of the capsule are water soluble substances.

Preferred methods for manufacture of such release capsules, preferred agents to be released, hydrophobic material and metal oxide materials will be described in detail hereinafter.

As mentioned above, a preferred release capsule of the present invention comprises a chlorodioxide generating agent as an agent to be released. It is a particularly surprising advantage that the present invention allows to effectively shield chlorodioxide generating agents from liquid water for a prolonged period of time, particularly allowing to delay the release of 50 wt.-% of the chlorodioxide generating agent from the capsule upon contact of the capsule with liquid water by at least 24 hours, preferably allowing to (a) delay the release of 75 wt.-% of the agent from the capsule upon contact of the capsule with liquid water by at least 24 h and, preferably, (b) delay the release of 50 wt.-% of the agent from the capsule upon contact of the capsule with liquid water by at least 48 h.

Chlorodioxide, also known as chlorine dioxide, is a known disinfectant as well as a strong oxidizing agent. It is particularly useful due to its bactericidal, algaecidal, fungicidal, bleaching and deodorizing properties; see U.S. Pat. No. 6,238,643. One of the advantages of chlorodioxide is that it may function without the formation of undesirable side products such as chloroamines or chlorinated organic compounds that otherwise can be produced when elemental chlorine is utilized for disinfection or fumigation. A disadvantage of chlorodioxide is that under standard conditions, i.e. 20° C., 1013 hPa, chlorodioxide is a gas and is thus difficult to store. To use chlorodioxide, this compound therefore frequently has to be generated in situ, as described for example in WO 2007/100531 A2 and WO 2007/149906 A2. However, chlorodioxide generating agents have to be handled with care, as chlorodioxide can explosively decompose into elementary chlorine and oxygen. Also, when forming chlorodioxide from salts it is mandatory to store chlorodioxide generating salt mixtures in a water-free form, lest the generation of chlorodioxide spontaneously starts, thereby leading to a loss of chlorodioxide or potentially to the formation of an explosive gas.

It has now surprisingly been found that release capsules of the present invention can be formed to encapsulate (salt) mixtures for the generation of chlorodioxide (herein termed “chlorodioxide generating agents”; such mixtures are e.g. sold under the trade name Aseptrol), and it is even more valuable that release capsules of the present invention can effectively shield encapsulated chlorodioxide generating mixtures from water. Thus, the invention allows providing a safe storage form for chlorodioxide generating agents, and in turn greatly widens the spectrum of potential applications for chlorodioxide.

Suitable chlorodioxide generating agents and substances thereof are e.g. described in WO 2007/100531 A2 and WO 2007/149906 A2 (see in particular aspects relating to “chlorine dioxide generating compositions” or “chlorine dioxide forming mixtures” respectively and corresponding constituents thereof).

A chlorodioxide generating agent for use in the present invention, comprising or consisting of two or more different substances (as described above), thus preferably comprises or consists of
one or more oxy-chlorine salts, preferably one or more oxy-chlorine salts as described in WO/2007/149906 A2, in particular one or more oxy-chlorine salts as described therein as preferred, in particular as described in the claims thereof, preferably a metal chlorite, and
an acid source, preferably an acid source as described in WO/2007/149906 A2, more preferably an acid source as described therein as preferred, in particular as described in the claims thereof,
(said oxy-chlorine salt(s), preferably said metal chlorite, and said acid source being able to react to form chlorine dioxide in water),
and optional further substances, namely
one or more free halogen sources, preferably one or more free halogen sources as described in WO/2007/149906 A2, more preferably one or more free halogen sources as described therein as preferred, in particular as described in the claims thereof,
and/or
one or more endothermic agents to mitigate exothermic reaction of the oxy-chlorine salt, preferably one or more endothermic agents as described in WO/2007/149906 A2, more preferably one or more endothermic agents as described therein as preferred, in particular as described in the claims thereof,
and/or
one or more phosphates, preferably one or more phosphates as described in WO/2007/100531 A2, more preferably one or more phosphates as described therein as preferred, in particular as described in the claims thereof,
and/or
one or more surfactants, preferably one or more surfactants as described in WO/2007/100531 A2, more preferably one or more surfactants as described therein as preferred,
and/or
one or more further substances, preferably one or more further substances described in WO 2007/100531 A2 and/or WO 2007/149906 A2 in connection with “chlorine dioxide generating compositions” or “chlorine dioxide forming mixtures” respectively.

The oxy-chlorine salts provide chlorine dioxide when the chlorine dioxide generating agent contacts water. An oxy-chlorine salt can be defined as one or more solid materials containing a chlorite anion, a chlorate anion, or a combination of chlorite and chlorate anions. In particular, the term oxy-chlorine salt can refer to one or more metal salts containing either or both chlorite or chlorate anions. Included in the term oxy-chlorine salt can be the individual salts, combined salts, and mixtures containing any combination of two or more individual and/or combined salts. In a preferred embodiment, the oxy-chlorine salts are water soluble.

Examples of metal chlorites include alkali metal chlorites such as lithium chlorite, sodium chlorite and potassium chlorite; and alkaline earth metal chlorites such as calcium chlorite and magnesium chlorite. In a preferred embodiment, the metal chlorite is sodium chlorite, dry technical grade sodium chlorite containing about 80% by weight of sodium chlorite and 20% by weight of other salts.

Suitable examples of metal chlorates include alkali metal chlorates such as sodium chlorate and potassium chlorate, and alkaline earth metal chlorates such as magnesium chlorate.

In a preferred embodiment, the acid source is a dry solid acid source. Examples of such dry solid acid sources include inorganic acid salts, such as sodium hydrogen sulfate and potassium hydrogen sulfate; salts containing anions of strong acids and cations of weak bases, such as aluminum chloride, aluminum nitrate, cerium nitrate, and iron sulfate; solid acids that can liberate protons into solution when contacted with water, for example a mixture of the acid ion exchanged form of molecular sieve ETS-10 (see U.S. Pat. No. 4,853,202) and sodium chloride; organic acids, such as citric acid and tartaric acid; and mixtures thereof. In a preferred embodiment, the solid acid source is a solid inorganic acid source, for example, sodium hydrogen sulfate. The amount of deposited metal oxide materials of a release capsule of the present invention, particularly where the agent to be released is a chlorodioxide generating agent, preferably is preferably less than 10 wt.-%, preferably less than 2 wt.-%, in particular 0.1 to 1 wt.-%, relative to the total mass of the capsule. It has now been found that at such amounts of metal oxides, small pores or irregularities of a hydrophobic shell material can be effectively sealed against influx of e.g. water into the release capsule which may otherwise lead to a dissolution of the agent to be released and its release into the aqueous environment.

The metal oxide to be deposited is preferably in the form of a powder, wherein powder is a material consisting of distinct particles with preferred average sizes of below 100 μm, preferably below 10 μm, which can be measured e.g. by standard laser diffraction measurements (e.g. from Malvern instruments), and is preferably chosen from the group consisting of ZnO, SnO2, SiO2, Al2O3, AlOOH, ZrO2, Fe2O3, MgO, CaO and TiO2, preferably from the group consisting ZnO, SiO2 and TiO2. The surface of such metal oxide (powder) can be made hydrophobic by e.g. attaching (fatty) acid groups to the surface, preferably by attaching a stearic acid group to the surface. Metal oxide materials suitable for the present invention may e.g. be hydrophobic treated ZnO or TiO2.

The metal oxide material is deposited in and/or on the shell of hydrophobic material. The hydrophobic material preferably is a paraffin, which advantageously imparts mechanical strength and water tightness to the release capsule, a wax material, a soft polymer (like short linear chained polyethylene or polyisobutylene) or a shellac. Preferably, the hydrophobic material comprises or consists of polyethylene wax, stearic acid wax, paraffin wax and/or fatty acid wax. Preferably, the hydrophobic material comprises or consists of a paraffin wax.

A release capsule of the present invention, preferably a release capsule as described above as preferred, preferably comprises a shell of paraffin wax (as described above), and further preferred has a zinc oxide material deposited in and/or on the shell (as described above).

Release capsules of the present invention, in particular release capsules comprising a chlorodioxide generating agent, can be used for bactericidal, algaecidal, fungicidal, bleaching and deodorizing purposes. Also, due to their encapsulation it is now possible to include e.g. chlorodioxide generating agents in cosmetic or pharmaceutical formulations for topical application to the human or animal body such that chlorodioxide is generated upon use of the formulation. For example, release capsules of the present invention comprising a chlorodioxide generating agent can be included in topically applied formulations like personal hygiene products, e.g. soaps, toothpastes, lozenges, disinfectant articles and wound dressings. When such formulation is topically applied, the shell of the particles of the present invention can be ruptured to release the chlorodioxide generating agent and expose it to the environment, thus generating chlorodioxide and making use of its bleaching and/or disinfecting properties.

Release capsules of the present invention, in particular release capsules comprising a chlorodioxide generating agent, can also be included in products not for topical application to the human or animal body. In particular, they can be used as water purification products, for the control of biofilm and non-cidal microorganism growth in water applications, e.g. swimming pools, water storage and waste water recycling, water ducts like pipes and hoses and commercial ice machines. Such capsules can be added to water pipes in green houses or cooling towers with an open to atmosphere cooling water circuit in order to avoid bio-fouling (growth of bioorganisms). In such a case, e.g. a released (generated) ClO2 limits the growth and reproduction of bioorganisms (for this application potassium based salts are of advantage, in particular such salts that do not hurt plant growth later on). Also, the release capsules of the present invention comprising a chlorodioxide generating agent can be used in dry form as a powder, particularly for surface sanitation, e.g. for carpets or laundry. In such applications, the release capsules can be ruptured mechanically, e.g. when walking on a carpet strewn with the release capsules, such that ambient moisture can react with the chlorodioxide generating formulation to generate chlorodioxide.

The content of the agent (or the agents respectively) to be released, preferably the chlorodioxide generating agent(s), is preferably at least 50 wt. % of the total mass of the core, more preferably at least 75 wt.-% and most preferably 90-100 wt.-%.

According to a preferred embodiment of the invention, the core amounts to at least 50 wt.-% of the total weight of the capsule, preferably to at least 66 wt.-%. Particularly preferred is a release capsule according to the invention that comprises or consists of a core (as described above) amounting to at least 50 wt.-%, preferably at least 66 wt.-% of the total weight of the capsule, a shell (as described above) amounting to 10 to 40 wt.-%, preferably 10 to 20 wt.-% of the total weight of the capsule, and metal oxide (as described above) amounting to 10 wt.-% or less, preferably to 2 wt.-% or less, in particular 0.1 to 1 wt.-%, of the total weight of the capsule.

As the agent to be released from a capsule of the present invention is effectively shielded from ambient water, according to one embodiment of the present invention the core of a release capsule of the present invention can advantageously have a low content of materials other than the agent to be released, for example further stabilizing matrix material. Thus, the present invention allows manufacturing particularly small capsules with a high content of the agent to be released.

Release capsules of the present invention, particularly those wherein the agent is a chlorodioxide generating agent, in particular those as herein described as preferred, have preferably a particle size that allows the capsules not being noticed as particles when used in a product (e.g. a creme or a paste), in particular not being noticed by the (human) tongue or skin, e.g. when consuming or touching the product. The release capsules of the present invention have preferably a size of 2000 μm or less, preferably of 1000 μm or less, more preferably of 500 μm or less, more preferably of 100 μm or less. In addition, the capsules have preferably a size of 1 μm or more. Thus, particularly preferred are sizes from 1 to 1000 μm, preferably from 1 to 500 μm, more preferably from 1 to 100 μm. More preferably, the capsules have a size of 10 μm or more. Thus, particularly preferred are sizes from 10 to 500 μm, in particular from 10 to 100 μm. The particle size is preferably measured by laser diffraction, e.g. Malvern Mastersize (cf. above). For a rough measurement, SEM and/or TEM may be used.

The invention also provides for a product comprising a release capsule of the present invention. Due to the water fastness of the release capsules of the present invention, such products can be solutions or dispersions in water, and can have a water content of up to 95 wt.-% of the total product, preferably 0-90 wt.-% and, for substantially dry products, preferably 0-10 wt.-%; in each case water content is given relative to the total weight of the product.

The products of the present invention can be cosmetic or pharmaceutical compositions, comprising a release capsule as described herein according to the invention, and a cosmetically and/or pharmaceutically acceptable carrier. Such compositions preferably are for topical application to the human or animal body. Particularly preferred cosmetic or pharmaceutical compositions are those wherein the release capsule(s) of the present invention comprises a chlorodioxide generating agent as an agent to be released. Such cosmetic or pharmaceutical compositions are particularly useful for bleaching, deodorizing and sanitation; corresponding preferred products have been described above.

According to the invention, there is also provided a method of manufacturing a release capsule, preferably a release capsule as described above, more preferably a release capsule as described above as particularly preferred, comprising the steps of
(i) providing a core having an agent to be eventually released, the agent comprising or consisting of two or more different substances (preferably as described above),
(ii) coating the core with a hydrophobic material, preferably a hydrophobic material as described above, to form a shell, and
(iii) depositing a metal oxide material, preferably a metal oxide material as described above, in and/or on the shell, wherein the amount of deposited metal oxide material is chosen to delay the release of 50 wt.-% of the agent (i.e. of the total weight/total content of the two or more different substances of the agent) from the capsule upon contact of the capsule with liquid water by at least 24 h.

The manufacturing method of the present invention as described particularly advantageously allows producing release capsules of the present invention in a coating process essentially in the absence of water. Thus, it is preferred that in the manufacturing method of the present invention the water content of the hydrophobic material in step (ii) is at most 10 wt.-%, preferably at most 3 wt.-%, more preferably at most 1 wt.-% and most preferably 0-0.5 wt.-%, relative to the total material used for coating the core.

In the manufacturing method of the present invention, preferred agents to be eventually released, preferred hydrophobic materials and preferred metal oxide materials are those as described above. With such materials it is possible to obtain the corresponding advantages. The above explanations relating to preferred amounts of the capsules' constituents—as well as the above definitions—apply accordingly.

Thus, e.g. the agent preferably comprises or consists of two or more different substances capable of generating a further substance upon contact with a gaseous or liquid medium (as described above), the further substance being different from the substances of the agent contained in the core of the capsule, preferably wherein the agent is a chlorodioxide generating agent.

The metal oxide material according to the manufacturing method of the present invention is deposited in and/or on the shell formed in step (ii). Preferably the metal oxide material is deposited in a sol-gel process or in a coacervation process. Such processes are useful for deposition of particularly fine metal oxide particles or layers, effectively sealing microscopic pores or cracks that may be present in the hydrophobic shell.

The deposition in step (iii) of the method of the present invention is preferably effected in a deposition medium comprising or consisting of a C1-C4 alcohol and/or water. It is particularly preferred to have a water content of at most 20 wt.-%, based on the total weight of deposition medium, preferably at most 60 wt.-%, more preferably at most 80 wt.-%. According to a preferred embodiment of the present invention the water content of the deposition medium is 100 wt.-%. An alternative preferred deposition medium comprises methanol, ethanol, n-propanol, isopropanol, butanol, isobutanol, tert-butanol or a mixture of two or more of these alcohols and optionally water, preferably water in an amount as described above. Of these, deposition media consisting of ethanol and water are particularly preferred, as trace amounts of ethanol potentially remaining attached to the thus produced release capsules can generally be considered not hazardous for further processing of the capsules of the present invention and particularly also in respect of the health of a user of a composition of the present invention comprising such release capsules.

The invention is hereinafter described in further detail by means of examples. It is to be noted that these examples are not intended to limit the meaning and scope of expressions otherwise used in this description or in the claims.

### EXAMPLES

### 1. Manufacturing of Release Capsules (Exemplary Manufacturing Instructions)

In the following, the manufacturing is performed with a model component-salt mixture of NaHCO3 and tartaric acid (as agent).
i. Granules of 200-500 μm are prepared as follows:

Powders (as defined above) of NaHCO3 and tartaric acid are added
a) to a mixer (e.g. Somakon mixer, where such is only an example for a number of potential mixers which can be used, such as plough share or paddles mixers, for example by Lödige, Ger. Ruberg, or ring shear mixers, for example by lödige, AVA-Hueb, Eirich mixers or similar mixers; all of them having in common a rotating mixing shaft (vertical or horizontal) equipped with mixing elements, which induce shear stresses into the powder bed, which is prepared in a mixing chamber), or
b) to a fluidized bed (a chamber with perforated bottom, where air is flowing through the perforated bottom at defined temperature).

To this mixing device (a) or b)), a suitable fluid and/or binder (e.g. water (when using water, the process is preferably performed at a temperature of 85° C.) and/or, if applicable, melted coating material (as defined and described above, preferably paraffin wax, e.g. material which is (also) used for the coating process later on)) is added, preferably through a nozzle and atomized by pressure or secondary heated air/nitrogen. If applicable, further constituents (e.g. one or more filler) may be added. During this operation the powder has to be continuously mixed at a defined speed. The mixing shear stresses and, if applicable, the amount of fluid and/or binder (as described above) influence the granule size. Additionally, heated air/nitrogen might be sent into the mixing chamber as fluidization gas or as a secondary gas in the mixer and/or the vessel of the mixing chamber might by heated. Via these two ways the powder bed is to have a temperature of below the melting temperature of the coating material, if applicable, preferably 5 to 20° C. below the melting temperature.
ii. The obtained granules are coated as follows:

After granule formation, the temperature of the powder bed may be reduced slightly (i.e. reduced by less than 20 K), if applicable, or kept at the same temperature to perform a surface coating step with a hydrophobic melt (as defined and described above, preferably paraffin wax). This is done in the same mixing device.

The (coated) granules are cooled outside the mixer or during slow operation of the mixer inside the mixing chamber.
iii. Metal oxide deposition in and/or on the shell:

If e.g. paraffin wax is used for coating (see above), the obtained granules/particles might be quite hydrophobic due to the paraffin wax coating and thus might need to be modified to be more hydrophilic (preferably with a wetting agent) in order to be able to conduct e.g. ZnO coating on the surface of these particles.

The wetting agents may be selected from BASF surfactants, ex-Ciba wetting agents and commercial surfactants with a small molecular weight, such as Aerosol OT, At-lox4913, Pluronic F127, Inutec SP1, Sokalan CP 20, Sokalan HP25, Lutrol F68, Sokalan HP80, Luvitec K80, Luvitec K17, Sokalan PM70, Sokalan PA80S and EFKA series. It was found that Aerosol OT and Sokalan HP25 could re-disperse paraffin wax coated particles well in water.

Preparation of the (e.g.) ZnO coating layer (as described above) may e.g. be carried out in pure ethanol, pure water or in a mixture of ethanol and water. Different reaction parameters may be tried in order to find an optimal reaction condition and obtain best quality of the products. The parameters to be varied are in particular (Zn2+-)salts (ZnCl2, ZnSO4, Zn(NO3)2, Zn(AC)2), ratios of Zn2+:OH— in M (1:2, 1:2.5, 1:3, 1:4), ratio of ethanol:water (pure ethanol, 1:1, 2:1, 4:1, 8:1, pure water), reaction time (5, 10, 15, 20, 25, 30 minutes), various polymers as an additive to accelerate precipitation and crystallization of ZnO (e.g. Sokalan PA, Sokalan HP series), introduction of multilayered (ZnO) deposition/coating (single, double, triple) and reaction temperature (20, 25, 30, 35, 40° C.).

The obtained products may be characterized by using XRD and REM. The release of agent(s) may be tested in water.

Particularly suitable results are obtained by the following trial: 0.5% Aerosol OT are introduced to the granules (in water), which is immediately followed by a ZnO coating process. ZnO coating is preferably conducted under the following conditions: ZnCl2:NaOH=2.5:1; 1% Sokalan PA 15 is added as an additive to accelerate crystallization of ZnO; reaction time=10 min; room temperature. The products are quickly dried in N2 atmosphere.

It is to be noted that the salts (agent) advantageously survive in the aqueous solution during the ZnO coating process.

### 2. (Model) Capsules According to the Present Invention

Capsules containing NaHCO3 and tartaric acid (as model agent) are produced according to the instructions described in example 1 above. The following substances have been used (based on a total weight of the capsule of 100 wt.-%., wherein Paraffin wax 1 and 2 may be the same or different waxes):

| | Total content |
|---|---|
| Substance | (wt.-%) |
| Core: | |
| Agent 1: NaHCO3 | 30 |
| Agent 2: Tartaric acid | 30 |
| Binder: Paraffin wax 1 (melting point 58° C. [symbol] | 3 |
| Filler: SiO2 Sipernat D17 | ad 100 |
| (Hydrophobic) Shell: | |
| Coating substance: Paraffin wax 2 (melting | 32 |
| point 58° C.) for coating | |
| Me-Oxide coating of shell: ZnO | <<1 |

[symbol]

indicates data missing or illegible when filed

Granule preparation was performed in a Somakon mixer (as described above), at 500 rpm and 50° C. for 5 min (spray nozzle Schlick pressure nozzle 0.8 mm, heated electrically).

The (wax) coating of granules was performed in a Somakon mixer (as described above) at 250 rpm and 45° C. for 10 min.

ZnO coating was performed according to above procedure (see example 1).

Release (here: decrease of mass of core: NaHCO3 and tartaric acid; assuming paraffin wax and ZnO do not dissolve in water) after immersion of capsules in water (10 g in 200 mL) was as follows:

| | |
|---|---|
| 20 min | <10 wt.-% release |
| 12 h | <40% wt.-% release |
| 24 h | <50% wt.-% release |

Release of CO2 was determined by chemical tritration.

### 3. Comparative Example

Capsules containing NaHCO3 and tartaric acid (as model agent) are produced according to the instructions described in example 1 above. However, no metal oxide material deposition was performed. The following substances have been used (based on a total weight of the capsule of 100 wt.-%., wherein Paraffin wax 1 and 2 may be the same or different waxes):

| | Total content |
|---|---|
| Substance | (wt.-%) |
| Core: | |
| Agent 1: NaHCO3 | 30 |
| Agent 2: Tartaric acid | 30 |
| Binder: Paraffin wax 1 (melting point 58° C.) | 3 |
| Filler: SiO2 Sipernat D17 | ad 100 |
| (Hydrophobic) Shell: | |
| Coating substance: Paraffin wax 2 (melting | 32 |
| point 58° C.) for coating | |
| Me-Oxide coating of shell: ZnO | — |

Granule preparation was performed in a Somakon mixer (as described above), at 500 rpm and 50° C. for 5 min (spray nozzle Schlick pressure nozzle 0.8 mm, heated electrically).

The (wax) coating of granules was performed in a Somakon mixer (as described above) at 250 rpm and 45° C. for 10 min.

No ZnO coating was applied.

Release (here: decrease of mass of core: NaHCO3 and tartaric acid; assuming paraffin wax and ZnO do not dissolve in water) after immersion of capsules in water (10 g in 200 mL) was as follows:

| | |
|---|---|
| 20 min | <50% wt.-% release |
| 12 h | >80% wt.-% release |
| 24 h | >90% wt.-% release |

Release of CO2 was determined by chemical tritration only in first 1 h.

1-15. (canceled)

16. A release capsule comprising a core having an agent to be eventually released, the agent comprising two or more different substances, the core being surrounded by a shell of a hydrophobic material and a metal oxide material deposited in and/or on the shell, wherein the amount of metal oxide material is effective to delay the release of 50 wt.-% of the agent from the capsule upon contact of the capsule with liquid water by at least 24 h.

17. The release capsule of claim 16, wherein the amount of metal oxide material is effective to (a) delay the release of 75 wt.-% of the agent from the capsule upon contact of the capsule with liquid water by at least 24 h and, preferably, (b) delay the release of 50 wt.-% of the agent from the capsule upon contact of the capsule with liquid water by at least 48 h.

18. The release capsule of claim 16, wherein the agent comprises two or more different substances capable of generating a further substance upon contact with a gaseous or liquid medium, the further substance being different from the substances of the agent contained in the core of the capsule, preferably wherein the agent is a chlorodioxide generating agent.

19. The release capsule of claim 16, wherein the agent contained in the core consists of two or more different, water soluble substances, preferably wherein all substances contained in the core of the capsule are water soluble substances.

20. The release capsule of claim 16, wherein the amount of deposited metal oxide material is 10 wt.-% or less, preferably 2 wt.-% or less, in particular 0.1 to 1 wt.-%, relative to the total mass of the capsule.

21. The release capsule of claim 16, wherein the capsule has a particle size of 1-2000 μm, preferably of 1-1000 μm, more preferably of 1-500 μm, more preferably of 10-100 μm.

22. A cosmetic or pharmaceutical composition comprising a release capsule of claim 16 and a cosmetically or pharmaceutically acceptable carrier, wherein the composition preferably is a composition for topical application to the human or animal body.

23. A method of manufacturing a release capsule, preferably a release capsule of claim 16, comprising the steps of
(i) providing a core having an agent to be eventually released, the agent comprising two or more different substances,
(ii) coating the core with a hydrophobic material to form a shell, and
(iii) depositing a metal oxide material in and/or on the shell, wherein the amount of deposited metal oxide material is chosen to delay the release of 50 wt.-% of the agent from the capsule upon contact of the capsule with liquid water by at least 24 h.

24. The method according to claim 23, wherein the agent comprises two or more different substances capable of generating a further substance upon contact with a gaseous or liquid medium, the further substance being different from the substances of the agent contained in the core of the capsule, preferably wherein the agent is a chlorodioxide generating agent.

25. The method according to claim 23, wherein the amount of deposited metal oxide material is 10 wt.-% or less, preferably 2 wt.-% or less, in particular 0.1 to 1 wt.-%, relative to the total mass of the capsule.

26. The method according to claim 23, wherein deposition in step (iii) is effected in a deposition medium comprising a C1-C4 alcohol and optionally water.

27. The release capsule of claim 16, obtained by the method of claim 23.

28. The release capsule of claim 16, for use in a method for treatment of the human or animal body by surgery or therapy, preferably for topical disinfection.

29. A chlorodioxide generating agent for use in a method for treatment of the human or animal body by surgery or therapy, preferably for topical disinfection.

30. Use of a medium comprising a C1-C4 alcohol and optionally water for deposition of a metal oxide in and/or on a hydrophobic shell material.
